(19) United States
(12) Patent Application Publication
(10) Pub. No.: US 2023/0406881 A1
(43) Pub. Date: Dec. 21, 2023

(54) BUFFERS AND METHODS FOR PURIFYING PROTEINS

(71) Applicant: ARES TRADING S.A., AUBONNE (CH)

(72) Inventors: NICOLAS-JULIAN HILBOLD, CORSIER-SUR-VEVEY (CH); VICTOR PASQUIER, CORSIER-SUR-VEVEY (CH); THIBAULT DUTRONC, CORSIER-SUR-VEVEY (CH)

(21) Appl. No.: 18/037,787

(22) PCT Filed: Nov. 20, 2020
(86) PCT No.: PCT/EP2020/082950

§ 371 Date: May 19, 2023

(51) Int. Cl. C07K 1/18; C07K 1/22; B01D 15/20; B01D 15/42; B01D 15/16

(52) U.S. Cl. CPC C07K 1/18; C07K 1/22; B01D 15/203; B01D 15/424; B01D 15/168

(57) ABSTRACT

The invention relates to the field of protein purification processes involving several chromatography steps. The invention pertains to a method for purifying a protein, preferably an antibody or fragment thereof or a protein containing said fragment, from a complex solution, wherein said method comprises at least two chromatography steps which are performed using buffers comprising or consisting of the same chemical compounds. The invention is particularly useful for large scale production and purification of recombinant proteins.

### FIELD OF INVENTION

The invention relates to the field of protein purification processes involving several chromatography steps. The invention pertains to a method for purifying a protein, preferably an antibody or fragment thereof or a protein containing said fragment, from a complex solution, wherein said method comprises at least two chromatography steps which are performed using buffers comprising or consisting of the same chemical compounds. The invention is particularly useful for large scale production and purification of recombinant proteins.

### BACKGROUND OF THE INVENTION

Recombinant proteins, and in particular antibodies or fusion proteins are generally purified from cell culture medium using multiple-steps resin chromatography processes, to separate the protein of interest from nucleic acids or other molecules present in the cell culture medium. The purification process typically comprises two or three steps of liquid chromatography, wherein a liquid mobile phase comprising the protein of interest is passed on a solid stationary phase, which may be for instance a resin or a membrane functionalized with specific chemicals, the solid stationary phase retaining molecules based on their physico-chemical properties. Proteins are made up of zwitterionic amino acid compounds; the net charge of a protein can be positive or negative depending on the pH of the environment. The specific isoelectric point (pI) of a protein can be used to determine whether its net charge will be positive or negative at a certain pH. This property as well as the hydrophobicity and size of the protein of interest are used to model the purification process around, for instance in choosing the specificity of the solid supports to be used for the chromatography steps. Further, buffers of various pH can be used to modify the net charge of the protein to be purified, and thereby increase or inhibit binding of said protein with the various supports used. In the field of antibody purification, the purification process typically starts with a “capture step”, using affinity chromatography, very commonly performed using a Protein A-based affinity chromatography, and which is intended to capture the antibody based on Protein A affinity for the Fc region. Alternative affinity chromatography based on Protein L, recombinant affinity ligands or synthetic affinity ligands may be used if more appropriate. In addition, this capture step is typically followed by polishing steps, intended to further separate the protein of interest from remaining contaminants such as DNA, HCP and endotoxins. Ion-Exchange (IEX) chromatography is widely used in the biopharmaceutical industry for polishing steps. IEX columns rely on electrostatic interactions between surface charges on molecules and charged functional groups on the solid stationary phase. In IEX, the binding characteristics between molecules present in the sample to be purified are determined by the net charge of the molecules, the charge distribution over the surface of the resin, the ligand type of the resin (strong and weak ion exchangers are available commercially), and pore size. IEX columns can be sorted into either cation exchange (CEX) and anion exchange (AEX) columns, according to their chemical properties. CEX typically use a negatively charged ion exchange solid support with an affinity for molecules having net positive surface charges, while AEX typically use a positively charged ion exchange solid support. Other types of chromatography, such as Hydrophobic-interaction chromatography (HIC), wherein the solid support comprises hydrophobic ligands and thus can be used to separate compounds based on their hydrophobic properties, may also be used. Mixed-mode chromatography (MMC), which relies on solid support combining 2 physico-chemical properties, is commonly used as part of the polishing steps.

Although this set up has proven very effective in providing highly purified proteins, each chromatography requires the use of several buffer solutions, in order to equilibrate the column, load the sample in appropriate chemical conditions, wash impurities, elute the protein of interest and regenerate the column. For any and each of these steps, a specific buffer must be formulated, for which pH and ionic strength must be adapted. Accordingly, conventional purification processes generally require the formulation and storage of numerous different buffers, which has an important impact in terms of time spend, space used and overall financial costs for a company.

There is therefore a need to streamline the provision and storage of chromatography buffers without compromising their buffering or chemical qualities or affecting chromatography performances.

### SUMMARY OF THE INVENTION

The invention provides aqueous buffers comprising substantially the same components, that can be used for all the common steps of chromatography purification of recombinant proteins, and thus allows for the simplification of large-scale purification processes. The buffers of the invention all contain acetate, phosphate, and TRIS base. They may be prepared from concentrated buffer solutions, also herein called mother buffer solutions, by simple dilution with purified water. In order to adapt conductivity and/or pH, sodium hydroxide and/or sodium chloride can further be added. The invention provides a simple buffer formulation with an extensive buffering region comprised between pH 2 and pH 9. In other terms, on this pH region the pH of the buffer solution upon addition of NaOH evolves according to a curve which is quasi-linear, without sudden pH rise. It is well known in the art that a buffer having a high conductivity, otherwise called high ionic strength, would interfere with binding of the protein of interest onto ion exchange columns, which may be undesirable. The aqueous buffers of the invention have been formulated so as possess a conductivity that, unless it is voluntarily increased by addition of NaCl, remains relatively low over the entire buffering region upon addition of NaOH, thereby providing buffer solutions that are not susceptible to affect negatively the quality of the purification of proteins.

Because of this very large buffer region, the buffers of the invention are very versatile, and can easily be used for any step of chromatography, from equilibrating the column, to elution of the protein of interest, including washing steps, and this independently from the type of chromatography used or protein to be purified. The buffers of the invention can be used as equilibration or rinse buffers, loading adjustment buffers, wash buffer, elution buffer and even as regeneration buffer, in any type of chromatography (affinity, ion-exchange, mixed-mode), and independent of whether said chromatography steps are performed in bind/elute mode or flow-through mode.

The invention therefore provides methods for purifying a protein of interest using chromatography, preferably liquid chromatography, wherein the chromatography steps rely essentially, or even completely, on the use of the buffers of the invention. In addition, the method set forth can be implemented so as to use, for the entire purification process, chromatography buffers which are all derived from the same concentrated mother by simple dilutions, and thereby have the same molar ratio of acetate over phosphate, and/or the same molar ratio of phosphate over Tris base, thereby simplifying process steps and reducing the need for storage space and the time required to prepare buffers.

### Definitions

In the context of the invention, the term “protein” shall be construed as generally understood in the art, that is to say as referring to a macromolecule comprising one or more long chains of amino acid residues. In the context of the invention, a protein typically contains at least 20 amino-acid residues and/or has a molecular weight of at least 15 kD. In the context of the invention, the term “protein” should be construed as generally understood in the field of protein production and purification, that is to say as referring broadly to compound composed essentially of amino-acids, and which may comprise post-translation modifications such as glycosylation. In the context of the invention, the protein may be for instance an antibody or a fragment thereof, a chimeric protein, an enzyme, or receptor protein.

In the context of the invention, the term “recombinant protein” shall be construed as generally understood in the art, that is to say as referring to a protein which is obtained by genetic engineering. Typically, a recombinant protein is produced by a host whose genetic material has been modified by genetic engineering. This definition also encompasses proteins produced in cell-free systems, and which production involves genetic engineering steps. This definition therefore encompasses proteins which sequence has been modified by recombinant means, as well as protein having a native sequence and produced in recombinant hosts.

The term “antibody” includes, inter alia, polyclonal antibodies, affinity-purified polyclonal antibodies, monoclonal antibodies, and antigen-binding fragments, such as F(ab′)2, Fab proteolytic fragments, and single chain variable region fragments (scFvs). Genetically engineered intact antibodies or fragments, such as chimeric antibodies, scFv and Fab fragments, as well as synthetic antigen-binding peptides and polypeptides, are also included.

The term “recombinant antibodies” means antibodies produced by recombinant technics. Because of the relevance of recombinant DNA techniques in the generation of antibodies, one needs not be confined to the sequences of amino acids found in natural antibodies; antibodies can be redesigned to obtain desired characteristics. The possible variations are many and range from the changing of just one or a few amino acids to the complete redesign of, for example, the variable domain or constant region. Changes in the constant region will, in general, be made in order to improve, reduce or alter characteristics, such as complement fixation (e.g. complement dependent cytotoxicity, CDC), interaction with Fc receptors, and other effector functions (e.g. antibody dependent cellular cytotoxicity, ADCC), pharmacokinetic properties (e.g. binding to the neonatal Fc receptor; FcRn). Changes in the variable domain will be made in order to improve the antigen binding characteristics. In addition to antibodies, immunoglobulins may exist in a variety of other forms including, for example, single-chain or Fv, Fab, and (Fab′)2, as well as diabodies, linear antibodies, multivalent or multispecific hybrid antibodies.

The term “antibody fragment” refers to a fragment of an intact or a full-length chain or antibody, usually the binding or variable region. Said portions, or fragments, should maintain at least one activity of the intact chain/antibody, i.e. they are “functional portions” or “functional fragments”. Should they maintain at least one activity, they preferably maintain the target binding property. Examples of antibody portions (or antibody fragments) include, but are not limited to, “single-chain Fv”, “single-chain antibodies.” “Fv” or “scFv”. These terms refer to antibody fragments that comprise the variable domains from both the heavy and light chains, but lack the constant regions, all within a single polypeptide chain. Generally, a single-chain antibody further comprises a polypeptide linker between the VH and VL domains which enables it to form the desired structure that would allow for antigen binding. In specific embodiments, single-chain antibodies can also be bi-specific and/or humanized.

A “Fab fragment” is comprised of one light chain and the variable and CH1 domains of one heavy chain. The heavy chain of a Fab molecule cannot form a disulfide bond with another heavy chain molecule. A “Fab′ fragment” that contains one light chain and one heavy chain and contains more of the constant region, between the CH1 and CH2 domains, such that an interchain disulfide bond can be formed between two heavy chains is called a F(ab′)2 molecule. A “F(ab′)2” contains two light chains and two heavy chains containing a portion of the constant region between the CH1 and CH2 domains, such that an interchain disulfide bond is formed between two heavy chains. Having defined some important terms, it is now possible to focus the attention on particular embodiments of the instant invention.

In the context of the invention, the term “isoelectric point”, also referred to as pI, H(I) or IEP, is the pH at which a particular molecule carries no net electrical charge or is electrically neutral in the statistical mean. The net charge on a molecule is affected by the pH of its surrounding environment and can become more positively or negatively charged due to the gain or loss, respectively, of protons (H+). The isoelectric point of a protein can be assessed by calculation based on the amino-acid sequence of the protein, or can be determined experimentally, for instance using the icIEF (Imaged Capillary Isoelectric Focusing). method disclosed in Goyon et al. As indicated in Goyon et al., experimental pI as determined using this method were very well correlated with calculated pIs, such that any of said method may be used by the person skilled in the art. Preferably, for the sake of clarity, the pI has defined herein correspond to the pI as determined by the icIEF (Imaged Capillary Isoelectric Focusing). method.

Typically, recombinant antibodies have an isoelectric point comprised between about 6 and 9.5 In the context of the invention, the term “complex solution” refers to an aqueous based solution comprising the protein of interest, and which may further comprise other compounds (also called impurities), such as for instance compounds typically present in a cell culture medium or residues of cell culture.

In the context of the invention, the term “chromatography” refers to a method based on phase equilibrium partitioning and aimed at separating compounds present in a complex mixture.

In the context of the invention, the term “liquid chromatography” refers to a chromatography method aimed at separating molecules present in a liquid mobile phase, by using a solid stationary phase, and in which which a solute of interest in a complex solution is separated from other solutes in said complex solution as a result of differences in rates at which the individual solutes of the complex solution migrate through said solid stationary phase under the influence of a liquid mobile phase. Typically, the solid stationary phase comprises or consists of material displaying physical and/or chemical properties that enable the retention of molecules within or at the surface of said material based on the physical or chemical features of said molecules, such as their size, polarity, affinity, hydrophile properties. Accordingly, the term “liquid chromatography” encompasses all liquid chromatography techniques known in the art such as affinity chromatography, size exclusion chromatography, ion exchange chromatography, hydrophobic interaction chromatography, multimodal or mixed mode chromatography. As is well known in the art, a chromatography method may be implemented by contacting the liquid mobile phase with a planar solid stationary phase, in which case it is usually referred to as planar chromatography, or it may be implemented by pouring the liquid mobile phase on a vertical stationary phase, in which case it is usually referred to as column chromatography.

In the context of the invention, a “liquid chromatography step” should be understood as referring to any of the steps of the chromatography method performed with the chromatography column such as for instance equilibrating the solid stationary phase, flowing of the protein-containing complex solution to be purified onto or through the solid stationary phase, washing the solid stationary phase, eluting the protein of interest from the solid stationary phase (when in bind/elute mode), and regenerating the solid stationary phase. In addition, should an optional step of diluting

In the context of the invention, the terms “bind-elute mode”, used in connection with a chromatography step or technique, refers to a mode of implementing a chromatography technique wherein the chromatography technique is set up so as to separate the protein to be purified from the other components of the liquid mobile phase by, in a first step, maximizing retention of the protein to be purified on the solid stationary phase while minimizing retention of other components of the liquid mobile phase on the solid stationary phase, and in a further separate step maximizing dissociation of the protein of interest from the solid stationary phase (thereby eluting it).

In the context of the invention, the terms “frontal”, used in connection with a chromatography step or technique, refers to a mode of implementation of a chromatography technique, wherein the chromatography technique is set up so as to separate the protein to be purified from the other components of the liquid mobile phase by minimizing retention of the protein to be purified on the solid stationary phase while retention of other components of the liquid mobile phase on the solid stationary phase. In this condition, the interaction between impurities and ligands of the solid stationary phase must be stronger than the interaction between the interest protein and ligands of the solid stationary phase. Ultimately, during the loading step, the impurities will replace the potentially bonded proteins of interest on the ligands allowing separation.

In the context of the invention, the terms “flow-through mode”, used in connection with a chromatography step or technique, refers to a mode of implementation of a chromatography technique, wherein the chromatography technique is set up so as to separate the protein to be purified from the other components of the liquid mobile phase by minimizing retention of the protein to be purified on the solid stationary phase while retention of other components of the liquid mobile phase on the solid stationary phase.

In the context of the invention, the term “equilibration buffer” is meant to refer to a buffer which does not contain the protein of interest, and is passed onto the solid phase of a chromatography column modify the pH or conductivity of solid stationary phase of the chromatography, prior to passing the sample containing the protein of interest on the solid stationary phase, so as to enable or improve the further separation of the protein of interest from other components of the sample. For instance, when used in bind/elute mode, wherein binding of the protein of interest to the solid phase is sought, the equilibration buffer is set to maximize further retention of the protein of interest on said solid phase while minimizing retention of the other components present in the liquid phase. Alternatively, when used in flow-through mode, wherein binding of the impurities present in the liquid phase is sought, the equilibration buffer is set to minimize further retention of the protein of interest on said solid phase while maximizing retention of the other components present in the liquid phase.

In the context of the invention, the term “loading adjustment buffer” is meant to refer to a buffer intended to dilute the liquid phase (containing the protein of interest) to be passed onto the solid phase of a chromatography column. Typically, loading adjustment buffers are used to modify the pH or conductivity of the liquid phase so as to enable or improve separation of the protein of interest from other components of the liquid phase by the solid phase. For instance, when used in bind/elute mode, wherein binding of the protein of interest to the solid phase is sought, the loading adjustment buffer is set to maximize retention of the protein of interest on said solid phase while minimizing retention of the other components present in the liquid phase. Alternatively, when used in flow-through mode, wherein binding of the impurities present in the liquid phase is sought, the loading adjustment buffer is set to minimize retention of the protein of interest on said solid phase while maximizing retention of the other components present in the liquid phase.

In the context of the invention, the term “wash buffer” refers to a buffer used in bind/elute mode, and which is intended to and appropriate for eluting impurities from the solid phase without eluting the protein of interest from the solid phase.

In the context of the invention, the term “elution buffer” refers to a buffer used in bind/elute mode, and which is intended to and appropriate for eluting the protein of interest from the solid phase. In the context of the invention, the term “regeneration buffer” refers to a buffer used on the solid phase, after said solid phase has been used to separate the protein of interest from the other components of the liquid phase, to remove from the solid support substantially all the compounds from the liquid phase.

In the context of the invention, the term “rinse buffer” or “re-equilibration buffer” is meant to refer to a buffer which does not contain the protein of interest, and is passed onto the solid phase of a chromatography column after said chromatography has been used to purify a liquid phase of interest, so as to modify the pH or conductivity of solid stationary phase of the chromatography, prior to passing a new sample on the solid stationary phase. Typically, the, “rinse buffer” or “re-equilibration buffer” has chemical properties similar to the equilibration buffer.

In the context of the invention, the term “affinity chromatography” refers to liquid chromatography method in which the solute of interest, such as for instance a protein of interest, is reversibly and specifically bound to a specific ligand immobilized on the solid stationary phase. Preferably, the specific ligand is covalently attached to the solid stationary phase and is accessible to the protein of interest in solution as the solution contacts the solid stationary phase. Binding of the protein of interest to the immobilized ligand allows separation from other components of the complex solution such as impurities which pass through the solid stationary phase while the protein of interest remains specifically bound to the immobilized ligand on the solid stationary phase. The specifically bound protein of interest is then removed from the immobilized ligand by modifying the chemical or physical conditions so as to affect the affinity constant. The binding of the protein of interest to the immobilized ligand may for instance be affected by modifying the pH using buffers having a low or a high pH, by modifying the electric charge of the protein of interest using buffers having a low or high conductivity, or by using competitive ligands. An affinity chromatography is typically perform using a bind/elute mode, comprising a first step wherein the conditions are set to maximize binding of the protein of interest to the specific ligand immobilized on the solid stationary phase, and a further step wherein the conditions are set to maximize dissociation of the protein of interest from the specific ligand immobilized on the solid stationary phase.

In the context of the invention, the term “Protein A” encompasses Protein A recovered from a native source thereof, Protein A produced synthetically (e.g. by peptide synthesis or by recombinant techniques), and variants thereof which retain the ability to bind proteins which have a CH2/CH3 domain, such as an Fc region.

In the context of the invention, the term “Protein L” encompasses Protein L recovered from a native source thereof, Protein L produced synthetically (e.g. by peptide synthesis or by recombinant techniques), and variants thereof which retain the ability to bind proteins which have a CH2/CH3 domain, such as an Fc region.

In the context of the invention, the term “ion exchange chromatography” or “IEX” refer to a liquid chromatography method aimed at separating ionizable molecules based on their total charge. This definition encompasses typically cation exchange chromatography and anion exchange chromatography. Both may be used either in bind/elute, frontal or flow-through mode, depending on the expected result.

A “cation exchange chromatography” or CEX refers to chromatography performed with a solid stationary phase which is negatively charged, and which thus has free cations which bind to positively charged molecules present in the solution passed over or through the solid phase. Cation exchange chromatography may for instance rely on the use of sulfanolate (strong cation exchanger) or carboxylate (weak cation exchanger) groups immobilized at the surface of the solid stationary phase

An “anion exchange chromatography” or AEX refers to chromatography performed with a solid stationary phase which is positively charged, and which thus has free anions which bind to negatively charged molecules present in the solution passed over or through the solid phase. Anion exchange chromatography may for instance rely on the use of diethyl-aminoethyl groups (DEAE) immobilized at the surface of the solid stationary phase.

In the context of the invention, the term “Hydrophobic-interaction chromatography” or HIC refers to a liquid chromatography method aimed at separating molecules based on their hydrophobic profile. Typically, a HIC is performed with a solid stationary phase which is characterized by hydrophobic ligands, interacting preferentially with hydrophobic regions of proteins present in the solution passed over or through the solid phase. HIC may for instance rely on the use of aliphatic chains (such as hexyl, butyl, phenyl groups) immobilized at the surface of the solid stationary phase.

In the context of the invention, the term “Multimodal chromatography” or “Mixed-Mode chromatography” or MMC refers to a liquid chromatography method aimed at separating molecules based on several physico-chemical properties of interest, such as for instance their total charge and their hydrophobic profile. Typically, MMC may be performed with a solid stationary phase which combines several physico-chemical properties of interest, such as for instance hydrophobic ligands and free ions. In MM, the solid stationary phase may for instance comprise a ligand combining several previously mentioned characteristics, or may comprise different types of individual ligands each having a characteristic of interest. The most common MMC relies on ligands containing ion exchanging chemical groups and aliphatic groups, leading to a combination of charged ligands and hydrophobic ligands, providing the so-called “bimodal” mode of interaction (in this example IEX- and HIC-based).

In the context of the invention, the term “aqueous buffer” refers to an aqueous-based solution displaying buffering properties, that has the ability to resist stricking pH changes due to the addition of an acid or a base by either absorbing or desorbing H+ and OH— ions, that is to say which pH evolves according to a curve which is roughly linear, without sudden pH rise further to the addition of either an acid or a base.

In the context of the invention, the term “TRIS base” refers to the compound 2-Amino-2-(hydroxymethyl)-1,3-propanediol, corresponding to CAS number 77-86-1.

### DETAILED DESCRIPTION OF THE INVENTION

The invention pertains to the use of aqueous buffers comprising or consisting of acetate, phosphate, TRIS base, and optionally sodium hydroxide and/or sodium chloride, for purifying a protein of interest from a complex solution, said use comprising least a first liquid chromatography and a second chromatography, wherein for each said liquid chromatography, said aqueous buffers are used as at least one buffer chosen among equilibration buffer, loading adjustment buffer, wash buffer, elution buffer, regeneration buffer and rinse buffer.

The invention further pertains to a method for purifying a protein of interest from a complex solution, said method comprising at least a first liquid chromatography and a second chromatography, wherein for each said liquid chromatography, aqueous buffers comprising acetate, phosphate, TRIS base, and optionally sodium hydroxide and/or sodium chloride are used as at least one buffer chosen among equilibration buffer, loading adjustment buffer, wash buffer, elution buffer, regeneration buffer and rinse buffer.

Advantageously, in said use or method, in either the first or the second liquid chromatography, said aqueous buffers are used as at least two buffers chosen among equilibration buffer, loading adjustment buffer, wash buffer, elution buffer, regeneration buffer and rinse buffer, preferably among equilibration buffer, wash buffer and regeneration buffer.

Advantageously, in said use or method, in the first and in the second liquid chromatography, said aqueous buffers are used as at least two buffers chosen among equilibration buffer, loading adjustment buffer, wash buffer, elution buffer, regeneration buffer and rinse buffer, preferably among equilibration buffer, wash buffer and regeneration buffer.

Advantageously, in said use or method, in either the first or the second liquid chromatography, said aqueous buffers are used as at least three buffers chosen among equilibration buffer, loading adjustment buffer, wash buffer, elution buffer, regeneration buffer and rinse buffer, preferably as equilibration buffer, wash buffer and regeneration buffer.

Advantageously, in said use or method, in the first and the second liquid chromatography, said aqueous buffers are used as at least three buffers chosen among equilibration buffer, loading adjustment buffer, wash buffer, elution buffer, regeneration buffer and rinse buffer, preferably as equilibration buffer, wash buffer and regeneration buffer.

Preferably, said use or method comprises a third chromatography. In those embodiments wherein the use or method according to the invention comprises a third chromatography, preferably at least an aqueous buffer aqueous buffer comprising acetate, phosphate, TRIS base, and optionally sodium hydroxide and/or sodium chloride, for said third liquid chromatography, as at least one buffer chosen among equilibration buffer, loading adjustment buffer, wash buffer, elution buffer, regeneration buffer and rinse buffer. Advantageously, in said use or method, for the first, the second and the third liquid chromatography, said aqueous buffers are used as at least three buffers chosen among equilibration buffer, loading adjustment buffer, wash buffer, elution buffer, regeneration buffer and rinse buffer, preferably as equilibration buffer, wash buffer and regeneration buffer.

The invention pertains to a method for purifying a protein of interest from a complex solution, wherein said method comprises purifying said complex solution containing said protein of interest through at least a first liquid chromatography and a second chromatography, wherein for each said first and second liquid chromatography, at least one step chosen from the list consisting of equilibrating said solid stationary phase, washing said solid stationary phase, eluting the protein of interest, and regenerating said solid stationary phase, is performed using aqueous buffers comprising the compounds acetate, phosphate, TRIS base, and optionally sodium hydroxide and/or sodium chloride.

Preferably, the invention pertains to a method for purifying a protein of interest from a complex solution, wherein said method comprises:
a) purifying said complex solution comprising said protein of interest using at least a first liquid chromatography comprising a first solid stationary phase, thereby obtaining a first protein of interest eluent, wherein at least one y step chosen from the list consisting of equilibrating said solid stationary phase, washing said solid stationary phase, eluting the protein of interest, and regenerating said solid stationary phase, is performed using aqueous buffers comprising the compounds acetate, phosphate, TRIS base, and optionally sodium hydroxide and/or sodium chloride; and
b) purifying the first protein of interest eluent of step a) using a second liquid chromatography comprising a second solid stationary phase, thereby obtaining a second protein of interest eluent, wherein at least one step chosen from the list consisting of equilibrating said solid stationary phase, washing said solid stationary phase, eluting the protein of interest, and regenerating said solid stationary phase, is performed using aqueous buffers comprising acetate, phosphate, TRIS base, and optionally sodium hydroxide and/or sodium chloride.

Optionally, in the method of the invention, prior to being flown through said second solid stationary phase, the first protein of interest eluent is mixed with a loading adjustment buffer according to the invention, that is to say an aqueous buffer comprising acetate, phosphate, TRIS base, and optionally sodium hydroxide and/or sodium chloride.

Preferably in the method of the invention detailed above, for either the first or the second liquid chromatography, at least two steps chosen from the list consisting of equilibrating said solid stationary phase, washing said solid stationary phase, eluting the protein of interest, and regenerating said solid stationary phase are performed using aqueous buffers comprising acetate, phosphate, TRIS base, and optionally sodium hydroxide and/or sodium chloride. Preferably in the method of the invention detailed above, in the first and the second liquid chromatography, at least two steps are performed using said aqueous buffers. Preferably in the method of the invention detailed above, in each of the first or the second liquid chromatography, all said steps among equilibrating said solid stationary phase, washing said solid stationary phase, eluting the protein of interest, and rinsing said solid stationary phase are performed using said aqueous buffers. Preferably in the method of the invention detailed above, in the first and the second liquid chromatography, all said steps among equilibrating said solid stationary phase, washing said solid stationary phase, eluting the protein of interest, and rinsing said solid stationary phase are performed using said aqueous buffers.

Preferably, said method comprises a third chromatography. In those embodiments wherein the method comprises a third chromatography, for each said first, second and third liquid chromatography, at least one step chosen in the list consisting of equilibrating said solid stationary phase, washing said solid stationary phase, eluting the protein of interest, and rinsing said solid stationary phase, is performed using said aqueous buffers.

Preferably, in any of the use and methods detailed herein, said aqueous buffers consist of acetate, phosphate, TRIS base, and optionally sodium hydroxide and/or sodium chloride. In the context of the invention, aqueous buffer contain water as the main solvent. In other terms, more preferably, in any of the use and methods detailed herein, said aqueous buffers consist of water, acetate, phosphate, TRIS base, and optionally sodium hydroxide and/or sodium chloride.

As indicated, the invention can further be implemented so as to simplify the process steps and reduce the need for storage space, by deriving all the buffers used according to the invention from a concentrated buffer solution, which can easily be diluted with water and adjusted for pH and conductivity using sodium hydroxide and/or sodium chloride. As a result, the uses and methods according to the invention are then performed using buffers as defined herein and which have the same molar ratio of phosphate over Tris base and the same molar ratio of acetate over phosphate.

Preferably, in any of the use and methods detailed herein, said aqueous buffers have the same molar ratio of phosphate over Tris base and the same molar ratio of acetate over phosphate.

The inventors have further determined value intervals for molar ratio of phosphate over Tris base as well as for molar ratio of acetate over phosphate which can be advantageously used to prepare versatile buffers, easily adaptable to any step of chromatography by simple means such as described above, i.e. by dilution with water and possible pH and conductivity adjustment using sodium hydroxide and/or sodium chloride.

Preferably, in any of the uses and methods detailed herein, the molar ratio of phosphate over Tris base in said aqueous buffers is comprised between 1 and 3, yet preferably between 1.5 and 2. Yet preferably, the molar ratio of phosphate over Tris is of about 2.

Preferably, in any of the uses and methods detailed herein, the molar ratio of acetate over phosphate in said aqueous buffers is comprised between 0.5 and 4, yet preferably between 1 and 2.5.

Preferably, in any of the use and methods detailed herein, the molar ratio of phosphate over Tris base in said aqueous buffers is comprised between 1.5 and 2. and the molar ratio of acetate over phosphate in said aqueous buffers is comprised between 1 and 2.5. Yet preferably, in any of the uses and methods detailed herein, the molar ratio of phosphate over Tris is of about 2, and the molar ratio of acetate over phosphate in said aqueous buffers is comprised between 1 and 2.5.

In a preferred embodiment of the uses and methods detailed herein, the molar ratio of phosphate over Tris is of about 2, and the molar ratio of acetate over phosphate in said aqueous buffers is of about 1. In another preferred embodiment of the use and methods detailed herein, the molar ratio of phosphate over Tris is of about 2, and the molar ratio of acetate over phosphate in said aqueous buffers is of about 2.5.

As the aqueous buffers to be used in the methods and uses detailed herein can be prepared from a single mother buffer solution, preferably in any of the use and methods detailed herein, the molar ratio of phosphate over Tris base in said aqueous buffers is comprised between 1.5 and 2. and the molar ratio of acetate over phosphate in said aqueous buffers is comprised between 1 and 2.5, and said aqueous buffers have the same molar ratio of phosphate over Tris base and the same molar ratio of acetate over phosphate.

Preferably, the protein of interest is a recombinant protein. Yet preferably, the protein of interest is an antibody, a fragment thereof, or a fusion protein comprising said fragment.

Preferably, the protein of interest has an isoelectric point comprised between 6 and 9.5, advantageously comprised between 6 and 8.5.

In the context of the invention, each of the first and second, and optionally the third, chromatography of the use and methods of the invention is chosen in the list consisting of affinity chromatography, cation-exchange chromatography, anion-exchange chromatography, an hydrophobic-interaction chromatography or a multimodal chromatography preferably having anion-exchange and hydrophobic-interaction properties. In the context of the invention, said chromatographies may be used in any order in the purification cascade. Any of said chromatographies may be used either in bind/elute, frontal or flow-through mode, depending on the expected result. Preferably, affinity chromatographies are performed in bind/elute mode. Preferably, ion-exchange chromatographies are performed in flow-through or frontal mode.

The preparation of chromatography buffers, and in particular adaptation of the pH and conductivity, depending on their intended use (equilibration, loading, wash, elution etc. . . . ), and according to the mode used (bind/elute or flow through) is well known in the art, and has been described thoroughly in many reference books and manuals. This general knowledge in the art has for instance been thoroughly discussed in Protein Chromatography (Giorgo Carta & Alois Jungbauer, Wiley-VCH Verlag GMBH, 2010) which the person skilled in the art could refer to.

Typically, when using cation exchange chromatography in bind/elute mode, the equilibration buffer will be formulated so as to have a pH superior to that of the pI of the groups immobilized on the solid stationary phase, so as to ensure that the solid stationary phase is negatively charged prior to flowing the sample containing the protein of interest. Then, if needed, the liquid fraction containing the protein of interest may be adjusted in pH and/or conductivity by using the different solutions available, so the proper interactions are improved. By doing so, the physico-chemical properties of the proteins will be modified by the solution environment, ensuring the proper interactions with the stationary phase. Typically, the pH can be decreased by using the adjustment solution below the pI of the protein of interest and above the pI of the ligand groups, so as to ensure that the protein of interest is charged positively and the ligand positively. loading adjustment buffer. Then, typically, the wash buffer(s) will be formulated so as to elute unwanted molecules without affecting the binding of the protein of interest to the solid stationary phase. Finally, typically, the elution buffer will be formulated so as to ensure that the protein of interest is released from the solid stationary phase, for instance by adapting the pH so that it is superior to the pI of the protein of interest and/or by adapting the conductivity of the elution buffer (in particular by increasing the concentration of positive ions, e.g. Na*).

Typically, when using cation exchange chromatography in flow-through mode, the equilibration buffer will be formulated so as to have a pH superior to that of the pI of the groups immobilized on the solid stationary phase, so as to ensure that the solid stationary phase is negatively charged prior to flowing the sample containing the protein of interest. Then, if needed, the protein of interest may be adjusted by a loading adjustment buffer which has a pH superior to the pI of the protein of interest, so as to ensure that the protein of interest has a negative charge and does not bind to the solid stationary phase.

Typically, when using anion exchange chromatography in bind/elute mode, the equilibration buffer will be formulated so as to have a pH inferior to that of the pI of the groups immobilized on the solid stationary phase, so as to ensure that the solid stationary phase is positively charged prior to flowing the sample containing the protein of interest. Then, if needed, the protein of interest may be diluted in a loading adjustment buffer which has a pH superior to the pI of the protein of interest, so as to ensure that the protein of interest has a negative charge and binds to the solid stationary phase. Then, typically, the wash buffer(s) will be formulated so as to elute unwanted molecules without affecting the binding of the protein of interest to the solid stationary phase. Finally, typically, the elution buffer will be formulated so as to ensure that the protein of interest is released from the solid stationary phase, for instance by adapting the pH so that it is inferior to the pI of the protein of interest and/or by adapting the conductivity of the elution buffer (in particular by increasing the concentration of negative ions, e.g. Cl⁻).

Typically, when using anion exchange chromatography in flow-through mode, the equilibration buffer will be formulated so as to have a pH inferior to that of the pI of the groups immobilized on the solid stationary phase, so as to ensure that the solid stationary phase is positively charged prior to flowing the sample containing the protein of interest. Then, if needed, the protein of interest may be diluted in a loading adjustment buffer which has a pH inferior to the pI of the protein of interest, so as to ensure that the protein of interest has a neutral or positive charge and does not bind to the solid stationary phase.

As already indicated, the specific composition of the buffers used in the methods detailed herein provides for a large range of possible buffers, such that the person skilled in the art will easily implement the use and methods of the invention by choosing and preparing aqueous buffers that are most adapted to the protein of interest to be purified, the intended use of the buffer, as well as the mode (such as bind/elute or flow through) in which the chromatographies are used. The person skilled in the art may therefore easily adapt the concentrations of the components of the buffer, as well as the pH and conductivity of said buffer, without using anymore compounds than those indicated.

Protein purification, and in particular antibody purification, is generally performed using affinity chromatography, which is very often performed either as the first chromatography step or at least in the first stages of the downstream purification cascade. As demonstrated in the experimental part, the aqueous buffers defined herein, which form an essential part of the uses and methods of the invention, have proven very efficient in downstream purification cascades involving affinity chromatography.

Preferably, in any of the use and methods detailed herein, at least the first or the second liquid chromatography is an affinity chromatography, yet preferably the first liquid chromatography an affinity chromatography. Preferably, the affinity chromatography is chosen in the list consisting of Protein A, Protein L and recombinant affinity ligands affinity chromatography. In other terms, preferably the affinity chromatograpghy is performed using a solid stationary phase comprising immobilized Protein A, Protein L or recombinant affinity ligands. Preferably, the affinity chromatography is a protein A affinity chromatography. In other terms, preferably the affinity chromatograpghy is performed using a solid stationary phase comprising immobilized protein A. Preferably, the affinity chromatography is performed in bind/elute mode.

Solid stationary phases comprising immobilized protein A are well known in the art and are commercially available. Commercialized stationary phases comprising immobilized protein A that could be used to implement the invention are for instance MabSelect™ resins commercialized by GE Healthcare Life Sciences (such as PrismA, SuRe™, SuRe LX, SuRe pcc®, Xtra™), Eshmuno® A and ProSep® Ultra Plus commercialized by Millipore, Amsphere™ A3 commercialized by JSR Life Sciences, Praesto® Jetted A50 commercialized by Purolite.

Protein purification, and in particular antibody purification, generally involves one or two ion exchange chromatographies, very often performed further to the affinity chromatography.

Preferably, in any of the use and methods detailed herein, at least one of the second and third liquid chromatography is an ion-exchange chromatography, yet preferably the second liquid chromatography is an ion-exchange chromatography. Yet preferably, in any of the use and methods detailed herein, the second and the third liquid chromatographies are ion-exchange chromatographies. Yet preferably, in any of the use and methods detailed herein, the second and the third liquid chromatographies are ion-exchange chromatographies, the second liquid chromatography and the third liquid chromatography are preferably performed in flow-through mode.

Preferably, in any of the use and methods detailed herein, at least one of the second and third liquid chromatography is a cation-exchange chromatography, yet preferably the second liquid chromatography is a cation-exchange chromatography. Preferably, the cation-exchange chromatography is performed in frontal or flow-through mode, yet preferably in flow-through mode.

Solid stationary phases adapted to cation-exchange chromatography are well known in the art and are commercially available. Commercialized solid stationary phases adapted to cation-exchange chromatography that could be used to implement the invention are for instance Fractogel® resins commercialized by Millipore (such as Fractogel® COO⁻, Fractogel® SO₃⁻), Eshmuno® resins commercialized by Millipore (such as Eshmuno® S, Eshmuno® HCX, Eshmuno® CPS, Eshmuno® CPX, Eshmuno® CP-FT), Sartobind S membranes commercialized by Sartorius, Mustang S membrane commercialized by Pall.

Preferably, in any of the use and methods detailed herein, at least one of the second and third liquid chromatography is an anion-exchange chromatography, yet preferably the third liquid chromatography is an anion-exchange chromatography. Preferably, the anion-exchange chromatography is performed in frontal or flow-through mode, yet preferably in flow-through mode.

Solid stationary phases adapted to anion-exchange chromatography are well known in the art and are commercially available. Commercialized solid stationary phases adapted to anion-exchange chromatography that could be used to implement the invention are for instance Eshmuno® Q resin commercialized by Millipore, Toyopearl® NH2-750F commercialized by Tosoh, Sartobind Q membrane commercialized by Sartorius, Mustang Q membrane commercialized by Pall, NatriFlo HD-Q membrane commercialized by Millipore.

Solid stationary phases adapted to multimodal chromatography are well known in the art and are commercially available. A commercialized solid stationary phase adapted to multimodal chromatography that could be used to implement the invention is for instance Capto-Adhere commercialized by GE Healthcare Life Sciences, which is a multimodal resin having anion-exchange and hydrophobic-interaction properties, Sartobind STIC commercialized by Sartorius

Preferably, in any of the use and methods detailed herein, said first liquid chromatography is an affinity chromatograpghy, preferably a protein A affinity chromatography, and said second chromatography is a cation-exchange chromatography, an anion-exchange chromatography, an hydrophobic-interaction chromatography or a multimodal chromatography preferably having anion-exchange and hydrophobic-interaction properties. Yet preferably, said first liquid chromatography is an affinity chromatograpghy, preferably a protein A affinity chromatography, and said second chromatography is a cation-exchange chromatography

Preferably, in any of the use and methods detailed herein, said first liquid chromatography is an affinity chromatograpghy, preferably a protein A affinity chromatography, and said second chromatography is a cation-exchange chromatography an anion-exchange chromatography, an hydrophobic-interaction chromatography or a multimodal chromatography preferably having anion-exchange and hydrophobic-interaction properties, preferably a cation-exchange chromatography, and said third chromatography is a cation-exchange chromatography an anion-exchange chromatography, an hydrophobic-interaction chromatography or a multimodal chromatography preferably having anion-exchange and hydrophobic-interaction properties, preferably an anion-exchange chromatography or a multimodal chromatography preferably having anion-exchange and hydrophobic-interaction properties.

Preferably, in any of the use and methods detailed herein, said first liquid chromatography is an affinity chromatograpghy, preferably a protein A affinity chromatography, and said affinity chromatography is performed in bind/elute mode; said second chromatography is a cation-exchange chromatography an anion-exchange chromatography, an hydrophobic-interaction chromatography or a multimodal chromatography preferably having anion-exchange and hydrophobic-interaction properties, preferably a cation-exchange chromatography, yet preferably a cation-exchange chromatography performed in frontal or flow-through mode, and said third chromatography is a cation-exchange chromatography an anion-exchange chromatography, an hydrophobic-interaction chromatography or a multimodal chromatography preferably having anion-exchange and hydrophobic-interaction properties, preferably an anion-exchange chromatography or a multimodal chromatography preferably having anion-exchange and hydrophobic-interaction properties, yet preferably an anion-exchange chromatography or a multimodal chromatography preferably having anion-exchange and hydrophobic-interaction properties performed in frontal or flow through mode.

More preferably, in any of the use and methods detailed herein:
the protein of interest is an antibody, a fragment thereof, or a fusion protein comprising said fragment;
the protein of interest has an isoelectric point comprised between 6 and 9.5;
the first liquid chromatography is an affinity chromatography, preferably a protein A affinity chromatography;
the second chromatography is a cation-exchange chromatography or an anion-exchange chromatography chromatography, preferably a cation-exchange chromatography;
the use or method optionally comprises a third chromatography which is a cation-exchange chromatography or an anion-exchange chromatography chromatography, preferably an anion-exchange chromatography chromatography;
for each said first and second, and optionally third, liquid chromatography, aqueous buffers comprising acetate, phosphate, TRIS base, and optionally sodium hydroxide and/or sodium chloride are used as at least one buffer chosen among equilibration buffer, loading adjustment buffer, wash buffer, elution buffer, and rinse buffer, preferably as at least three buffers chosen among equilibration buffer, loading adjustment buffer, wash buffer, elution buffer, and rinse buffer, yet preferably as equilibration buffer, wash buffer; elution buffer and alternatively regeneration buffer;
the molar ratio of phosphate over Tris base in said aqueous buffers is comprised between 1.5 and 2. and the molar ratio of acetate over phosphate in said aqueous buffers is comprised between 1 and 2.5, and said aqueous buffers have the same molar ratio of phosphate over Tris base and the same molar ratio of acetate over phosphate.

Yet preferably, in any of the use and methods detailed herein:
the protein of interest is an antibody, a fragment thereof, or a fusion protein comprising said fragment;
the protein of interest has an isoelectric point comprised between 6 and 9.5;
the first liquid chromatography is a protein A affinity chromatography;
the second chromatography is a cation-exchange chromatography;
the use or method comprises a third chromatography which is an anion-exchange chromatography;
for each said first, second and third liquid chromatography, aqueous buffers comprising acetate, phosphate, TRIS base, and optionally sodium hydroxide and/or sodium chloride are used as loading adjustment buffer, wash buffer, elution buffer and alternatively regeneration buffer;
the molar ratio of phosphate over Tris base in said aqueous buffers is of about 2, the molar ratio of acetate over phosphate in said aqueous buffers is comprised between 1 and 2.5, and said aqueous buffers have the same molar ratio of phosphate over Tris base and the same molar ratio of acetate over phosphate.

Yet preferably, in any of the use and methods detailed herein:
the protein of interest is an antibody, a fragment thereof, or a fusion protein comprising said fragment;
the protein of interest has an isoelectric point comprised between 6 and 9.5;
the first liquid chromatography is a protein A affinity chromatography and is performed in bind/elute mode;
the second chromatography is a cation-exchange chromatography and is preferably performed in frontal or flow-through mode;
the use or method comprises a third chromatography which is an anion-exchange chromatography and is preferably performed in frontal or flow-through mode;
for each said first, second and third liquid chromatography, aqueous buffers comprising acetate, phosphate, TRIS base, and optionally sodium hydroxide and/or sodium chloride are used as equilibration buffer, wash buffer, and elution buffer;
the molar ratio of phosphate over Tris base in said aqueous buffers is of about 2, the molar ratio of acetate over phosphate in said aqueous buffers is comprised between 1 and 2.5, and said aqueous buffers have the same molar ratio of phosphate over Tris base and the same molar ratio of acetate over phosphate.

Preferably, the methods detailed herein further comprise a low pH inactivation step, which may be implemented prior any of the liquid chromatographies, between two liquid chromatographies, or after all liquid chromatographies have been performed. Preferably, said low pH inactivation step is performed between the first liquid chromatography and the second liquid chromatography. Preferably, said low pH inactivation step is performed between the second liquid chromatography and the third liquid chromatography.

Preferably, the method of the invention further comprises a nanofiltration step and/or an ultrafiltration and diafiltration step, which may be implemented prior any of the liquid chromatographies, between two liquid chromatographies, or after all liquid chromatographies have been performed. Yet preferably, said nanofiltration step and/or an ultrafiltration and diafiltration step is performed after all liquid chromatographies.

### DESCRIPTION OF THE FIGURES

FIG. 1: Principle of buffer formulation and uses according to the invention

FIG. 2: Theorical titration curves with two different formulation using NaOH 1 mol/L

FIG. 3: Experimental titration curves. Evolution of pH and conductivity of an aqueous buffer solution comprising 55 mM acetate, 20 mM phosphate and 10 mM Tris base upon addition of gradual amount of NaOH

### EXAMPLES

The following examples are meant as an illustration of various embodiments of the invention and shall not be construed as limiting the scope or the definition of the invention in any way.

### Example 1. Theoretical and Experimental Titration of Two Buffers According to the Invention Support Versatility of Such Buffer Composition for DSP Processes

In the following experiments, titration of two aqueous buffers comprising 55 mM acetate, 20 mM phosphate and 10 mM Tris base or 20 mM acetate, 20 mM phosphate and 10 mM Tris base, upon addition of NaOH was performed first theoretically, i.e in silico using mathematical models defined in the art, or experimentally i.e. in laboratory settings.

1. Material and Methods

Solutions

For the preparation of the buffer, the following solutions were made:
55 mM acetate, 20 mM phosphate and 10 mM Tris base
NaOH 1M as titration solution
These solutions are made with the corresponding commercial species from Merck KGaA:
Glacial acetic acid (64-19-7)
Orthophosphoric acid 85% (7664-38-2)
Tris base (77-86-1)
NaOH 32% (1310-73-2)

Buffers are prepared at room temperature.

Theoretical Titration

The theoretical titration of two aqueous buffers comprising 55 mM acetate, 20 mM phosphate and 10 mM Tris base or 20 mM acetate, 20 mM phosphate and 10 mM Tris base, upon addition of NaOH is performed based on the Henderson-Hasselbalch equation (thermodynamic model):

$$pH = pKa + log ( \frac{[ A - ]}{[ HA ]} )$$

pH: pH of the solution for a given values of species concentration
Ka: acid dissociation constant
[A−]: concentration of the conjugate base
[HA]: concentration of the acid

An in-house software is used to perform the calculation for a multi-equilibrium case. This method is detailed by Baeza-Baeza et al. (Systematic Approach To Calculate the Concentration of Chemical Species in Multi-Equilibrium Problems, Journal of Chemical Education 2011 88 (2), 169-173).

Experimental Titration

Titration of the pH was performed using 713 pH Meter device from Metrohm.

Conductivity measurements was performed using 712 Conductometer from Metrohm.

Measurements are performed according supplier recommendations. Each point of the titration corresponds to a certain volume addition of NaOH 1M and thus, a total concentration of NaOH in the buffer.

Results

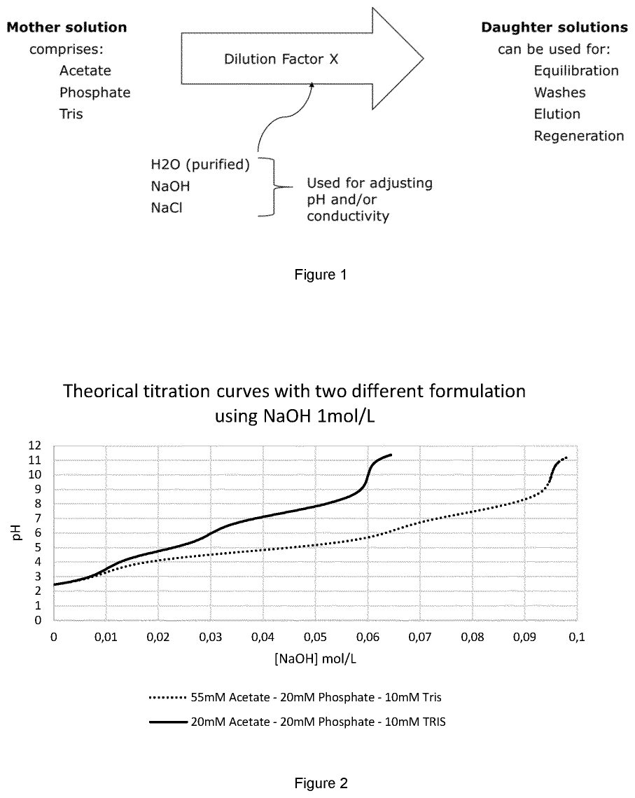

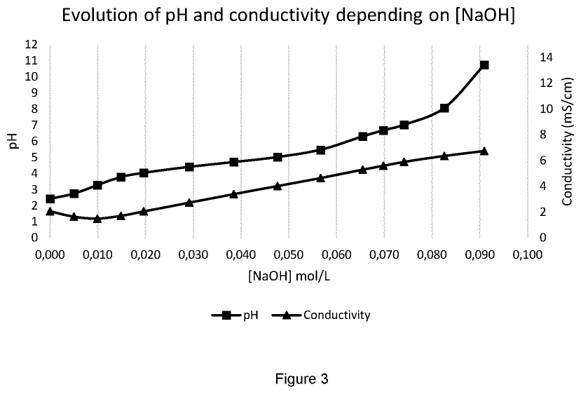

As shown in FIGS. 1 and 2, upon addition of NaOH, the pH of the aqueous buffer solution roughly evolves according to a linear curve. No striking pH rise is observed, confirming the buffer properties of the solution over the entire range comprised between pH 2 and 10.

### Example 2. Example of an Implementation of a Purification Cascade Using the Method of the Invention

A monoclonal IgG1 antibody (hereafter A1) having an isoelectric point of about 8.5 was recombinantly produced in CHO cells using usual cell culture methods, and the respective harvested cell culture fluids were purified according to two different embodiments of the method of the invention.

A. Purification Method 1

For purification method 1, the following downstream purification cascade was performed, in that order:
1. Protein A affinity chromatography using Amsphere A3 column (from JSR Life Science)
   a) The column was equilibrated using a buffer consisting of water, 0.05M Acetate, 0.02M Phosphate, 0.01M Tris Base, adjusted at pH 8 with NaOH 1M
   b) The cell culture harvest was filtrated on Stericup 0.22 μm from Merck Millipore then loaded on the column;
   c) A first wash buffer was flown through the column, which consisted of water, 0.05M Acetate, 0.02M Phosphate, 0.01 M Tris Base, adjusted at pH 8 with NaOH 1M
   d) A second wash buffer was flown through the column, which consisted of water, 0.05M Acetate, 0.02M Phosphate, 0.01 M Tris Base, 1.5M NaCl, adjusted at pH 5.5 with NaOH 1M
   e) A third wash buffer was flown through the column, which consisted of water, 0.05M Acetate, 0.02M Phosphate, 0.01 M Tris Base, adjusted at pH 8 with NaOH 1M
   f) The protein of interest was eluted from the column using a buffer which consisted of water, 0.05M Acetate, 0.02M Phosphate, 0.01 M Tris Base, adjusted at pH 3.2 with NaOH 1M. The eluate was conserved for the next chromatography;
   g) Regeneration was performed using NaOH 0.1M
   h) Sanitation was performed using NaOH 0.5M
   i) Rinse was performed using a buffer which consisted of water, 0.05M Acetate, 0.02M Phosphate, 0.01 M Tris Base, adjusted at pH 8 with NaOH 1M
2. CEX chromatography using Fractogel® EMD COO⁻ (M) column (from Merck Millipore);
   a) The column was equilibrated using a buffer consisting of water, 0.05M Acetate, 0.02M Phosphate, 0.01 M Tris Base, adjusted at pH 5.5 with NaOH 1M
   b) The eluate of step 1. f) was adjusted at pH 5.5 with NaOH 1M prior to the load on the column.
   c) A wash buffer was flown through the column, which consisted of water, 0.05M Acetate, 0.02M Phosphate, 0.01 M Tris Base, adjusted at pH 5.5 with NaOH 1M. The resulting flowthrough (2.b and 2.c) was conserved for the next chromatography;
   d) Regeneration was performed using a buffer which consisted of water, 0.05M Acetate, 0.02M Phosphate, 0.01 M Tris Base, 1M NaCl, adjusted at pH 8 with NaOH 1M
   e) Sanitation was performed using NaOH 0.5M
   f) Rinse was performed using a buffer which consisted of water, 0.05M Acetate, 0.02M Phosphate, 0.01 M Tris Base, adjusted at pH 5.5 with NaOH 1M
3. AEX chromatography using Capto Adhere column (from GE Healthcare);
   a) The column was equilibrated using a buffer consisting of water, 0.025M Acetate, 0.01M Phosphate, 0.005 M Tris Base, adjusted at pH 7.5 with NaOH 1M
   b) The CEX flowthrough was adjusted at pH 7.5 with NaOH 1M prior to the load on the column.
   c) A wash buffer was flown through the column, which consisted of water, 0.025M Acetate, 0.01M Phosphate, 0.005 M Tris Base, adjusted at pH 7.5 with NaOH 1M. The resulting flowthrough (3.b and 3.c) was conserved for analyses;
   d) Regeneration was performed using a buffer which consisted of water, 0.05M Acetate, 0.02M Phosphate, 0.01 M Tris Base, 1.5M NaCl, adjusted at pH 5.5 with NaOH 1M
   e) Sanitation was performed using NaOH 0.5M

Quality and impurities analysis for the flowthrough of step 3b)c) was performed. The results are presented in table 1.

B. Purification Method 2

For purification method 2, the following downstream purification cascade was performed, in that order:
1. Protein A affinity chromatography using Amsphere A3 column (from JSR Life Science)
   a) The column was equilibrated using a buffer consisting of water, 0.02M Acetate, 0.02M Phosphate, 0.01 M Tris Base, adjusted at pH 8 with NaOH 1M
   b) The cell culture harvest was filtrated on Stericup 0.22 μm from Merck Millipore then loaded on the column;
   c) A first wash buffer was flown through the column, which consisted of water, 0.02M Acetate, 0.02M Phosphate, 0.01 M Tris Base, adjusted at pH 8 with NaOH 1M
   d) A second wash buffer was flown through the column, which consisted of water, 0.02M Acetate, 0.02M Phosphate, 0.01 M Tris Base, 1.5M NaCl, adjusted at pH 5.5 with NaOH 1M
   e) A third wash buffer was flown through the column, which consisted of water, 0.02M Acetate, 0.02M Phosphate, 0.01 M Tris Base, adjusted at pH 8 with NaOH 1M
   f) The protein of interest was eluted from the column using a buffer which consisted of water, 0.02M Acetate, 0.02M Phosphate, 0.01 M Tris Base, adjusted at pH 3.2 with NaOH 1M. The eluate was conserved for the next chromatography;
   g) Regeneration was performed using NaOH 0.1M
   h) Sanitation was performed using NaOH 0.5M
   i) Rinse was performed using a buffer which consisted of water, 0.02M Acetate, 0.02M Phosphate, 0.01 M Tris Base, adjusted at pH 8 with NaOH 1M
2. CEX chromatography using Fractogel® EMD COO⁻ (M) column (from Merck Millipore);
   a) The column was equilibrated using a buffer consisting of water, 0.02M Acetate, 0.02M Phosphate, 0.01 M Tris Base, adjusted at pH 5.5 with NaOH 1M
   b) The eluate of step 1. f) was adjusted at pH 5.5 with NaOH 1M prior to the load on the column.
   c) A wash buffer was flown through the column, which consisted of water, 0.02M Acetate, 0.02M Phosphate, 0.01 M Tris Base, adjusted at pH 5.5 with NaOH 1M. The resulting flowthrough (2.b and 2.c) was conserved for the next chromatography;
   d) Regeneration was performed using a buffer which consisted of water, 0.02M Acetate, 0.02M Phosphate, 0.01 M Tris Base, 1M NaCl, adjusted at pH 8 with NaOH 1M
   e) Sanitation was performed using NaOH 0.5M
   f) Rinse was performed using a buffer which consisted of water, 0.02M Acetate, 0.02M Phosphate, 0.01 M Tris Base, adjusted at pH 5.5 with NaOH 1M
3. AEX chromatography using Capto Adhere column (from GE Healthcare);
   a) The column was equilibrated using a buffer consisting of water, 0.02M Acetate, 0.02M Phosphate, 0.01 M Tris Base, adjusted at pH 7.5 with NaOH 1M
   b) The CEX flowthrough was adjusted at pH 7.5 with NaOH 1M prior to the load on the column. c) A wash buffer was flown through the column, which consisted of water, 0.02M Acetate,
   0.02M Phosphate, 0.01 M Tris Base, adjusted at pH 7.5 with NaOH 1M. The resulting flowthrough (3.b and 3.c) was conserved for analyses;
d) Regeneration was performed using a buffer which consisted of water, 0.1M Acetate, 0.1M Phosphate, 0.05 M Tris Base
e) Sanitation was performed using NaOH 0.5M
f) Rinse was performed using a buffer which consisted of water, 0.05M Acetate, 0.02M Phosphate, 0.01 M Tris Base, adjusted at pH 7.5 with NaOH 1M

Quality and impurities analysis for the flowthrough of step 3b)c) was performed. The results are presented in table 2.

TABLE 1
A1 purification specifications further
to purification according to method 1

| | Buffer Mix 1 | | | | | |
|---|---|---|---|---|---|---|
| | Yield | Cumulative | HCP | DNA | HMW | LMW |
| Steps | % | yield % | ppm | ppb | % | % |
| Harvest | — | — | 212690 | 148234 | 4.8 | 2.7 |
| Capture | 97.1 | 97.1 | 7354 | 1893 | 9.2 | 2.6 |
| CEX | 88.2 | 85.6 | 3214 | | 2.5 | 2.3 |
| MM | 85.3 | 73.1 | 11 | | 0.3 | 1.8 |

TABLE 2
A1 purification specifications further
to purification according to method 2

| | Buffer Mix 2 | | | | | |
|---|---|---|---|---|---|---|
| | Yield | Cumulative | HCP | DNA | HMW | LMW |
| Steps | % | yield % | ppm | ppb | % | % |
| Harvest | | | 209462 | 2136078 | 3.5 | 6.6 |
| Capture | 100 | 100 | 4614 | 4996 | 7.5 | 7.0 |
| CEX | 87.7 | 87.7 | 1808 | — | 2.0 | 6.3 |
| MM | 78.3 | 68.7 | 20 | — | 0.3 | 4.7 |

Conclusion: The yield of both purification cascades are comparable (around 70%). Despite the use of different harvest lot, the aggregate percentage and HOP concentration are very low and comparable for both cascades (less than 20 ppm of HOP and 0.3% of HMW at the end of the purification). The high value of clipped form in the second example is due to the initial value of the corresponding harvest and a limited impact of the purification on the LMW. To conclude, the different buffer ratio used show great and comparable result in term of performance and purification.

### Example 3. Example of an Implementation of a Purification Cascade Using the Method of the Invention

A fusion protein comprising an antibody fragment (hereafter A2) having an isoelectric point of 6.09, was recombinantly produced in CHO cells using usual cell culture methods, and the respective harvested cell culture fluids were purified according to two embodiments of method of the invention.

A. Purification Method 1

For purification method 1, the following downstream purification cascade was performed, in that order:
1. Protein A affinity chromatography using Amsphere A3 column (from JSR Life Science)
   a) The column was equilibrated using a buffer consisting of water, 0.05M Acetate, 0.02M Phosphate, 0.01 M Tris Base, adjusted at pH 8 with NaOH 1M
   b) The cell culture harvest was filtrated on Stericup 0.22 μm from Merck Millipore then loaded on the column;
   c) A first wash buffer was flown through the column, which consisted of water, 0.05M Acetate, 0.02M Phosphate, 0.01 M Tris Base, adjusted at pH 8 with NaOH 1M
   d) A second wash buffer was flown through the column, which consisted of water, 0.05M Acetate, 0.02M Phosphate, 0.01 M Tris Base, 1.5M NaCl, adjusted at pH 5.5 with NaOH 1M
   e) A third wash buffer was flown through the column, which consisted of water, 0.05M Acetate, 0.02M Phosphate, 0.01 M Tris Base, adjusted at pH 8 with NaOH 1M
   f) The protein of interest was eluted from the column using a buffer which consisted of water, 0.05M Acetate, 0.02M Phosphate, 0.01 M Tris Base, adjusted at pH 3.2 with NaOH 1M. The eluate was conserved for the next chromatography;
   g) Regeneration was performed using NaOH 0.1M
   h) Sanitation was performed using NaOH 0.5M
   i) Rinse was performed using a buffer which consisted of water, 0.05M Acetate, 0.02M Phosphate, 0.01 M Tris Base, adjusted at pH 8 with NaOH 1M
2. CEX chromatography using Fractogel® EMD COO⁻ (M) column (from Merck Millipore);
   a) The column was equilibrated using a buffer consisting of water, 0.05M Acetate, 0.02M Phosphate, 0.01 M Tris Base, adjusted at pH 5.5 with NaOH 1M
   b) The eluate of step 1. f) was adjusted at pH 5.5 with NaOH 1M prior to the load on the column.
   c) A wash buffer was flown through the column, which consisted of water, 0.05M Acetate, 0.02M Phosphate, 0.01 M Tris Base, adjusted at pH 5.5 with NaOH 1M. The resulting flowthrough (2.b and 2.c) was conserved for the next chromatography;
   d) Regeneration was performed using a buffer which consisted of water, 0.05M Acetate, 0.02M Phosphate, 0.01 M Tris Base, 1M NaCl, adjusted at pH 8 with NaOH 1M
   e) Sanitation was performed using NaOH 0.5M
   f) Rinse was performed using a buffer which consisted of water, 0.05M Acetate, 0.02M Phosphate, 0.01 M Tris Base, adjusted at pH 5.5 with NaOH 1M
3. AEX chromatography using Eshmuno Q column (from Merck Millipore);
   a) The column was equilibrated using a buffer consisting of water, 0.025M Acetate, 0.01M Phosphate, 0.005 M Tris Base, adjusted at pH 5 with NaOH 1M
   b) The CEX flowthrough was adjusted at pH 5 with Acetic acid 1M prior to the load on the column.
   c) A wash buffer was flown through the column, which consisted of water, 0.025M Acetate, 0.01M Phosphate, 0.005 M Tris Base, adjusted at pH 5 with NaOH 1M. The resulting flowthrough (3.b and 3.c) was conserved for analyses;
   d) Regeneration was performed using a buffer which consisted of water, 0.05M Acetate, 0.02M Phosphate, 0.01 M Tris Base, 1.5M NaCl, adjusted at pH 5.5 with NaOH 1M
   e) Sanitation was performed using NaOH 0.5M
   f) Rinse was performed using a buffer which consisted of water, 0.05M Acetate, 0.02M Phosphate, 0.01 M Tris Base, adjusted at pH 5 with NaOH 1M Quality and impurities analysis for the flowthrough of step 3b)c) was performed. The results are presented in table 1.

C. Purification Method 2

For purification method 2, the following downstream purification cascade was performed, in that order:
1. Protein A affinity chromatography using Amsphere A3 column (from JSR Life Science)
   a) The column was equilibrated using a buffer consisting of water, 0.02M Acetate, 0.02M Phosphate, 0.01 M Tris Base, adjusted at pH 8 with NaOH 1M
   b) The cell culture harvest was filtrated on Stericup 0.22 μm from Merck Millipore then loaded on the column;
   c) A first wash buffer was flown through the column, which consisted of water, 0.02M Acetate, 0.02M Phosphate, 0.01 M Tris Base, adjusted at pH 8 with NaOH 1M
   d) A second wash buffer was flown through the column, which consisted of water, 0.02M Acetate, 0.02M Phosphate, 0.01 M Tris Base, 1.5M NaCl, adjusted at pH 5.5 with NaOH 1M
   e) A third wash buffer was flown through the column, which consisted of water, 0.02M Acetate, 0.02M Phosphate, 0.01 M Tris Base, adjusted at pH 8 with NaOH 1M
   f) The protein of interest was eluted from the column using a buffer which consisted of water, 0.02M Acetate, 0.02M Phosphate, 0.01 M Tris Base, adjusted at pH 3.2 with NaOH 1M. The eluate was conserved for the next chromatography;
   g) Regeneration was performed using NaOH 0.1M
   h) Sanitation was performed using NaOH 0.5M
   i) Rinse was performed using a buffer which consisted of water, 0.02M Acetate, 0.02M Phosphate, 0.01 M Tris Base, adjusted at pH 8 with NaOH 1M
2. CEX chromatography using Fractogel® EMD COO⁻ (M) column (from Merck Millipore);
   a) The column was equilibrated using a buffer consisting of water, 0.02M Acetate, 0.02M Phosphate, 0.01 M Tris Base, adjusted at pH 5.5 with NaOH 1M
   b) The eluate of step 1. f) was adjusted at pH 5.5 with NaOH 1M prior to the load on the column.
   c) A wash buffer was flown through the column, which consisted of water, 0.02M Acetate, 0.02M Phosphate, 0.01 M Tris Base, adjusted at pH 5.5 with NaOH 1M. The resulting flowthrough (2.b and 2.c) was conserved for the next chromatography;
   d) Regeneration was performed using a buffer which consisted of water, 0.02M Acetate, 0.02M Phosphate, 0.01 M Tris Base, 1M NaCl, adjusted at pH 8 with NaOH 1M
   e) Sanitation was performed using NaOH 0.5M
   f) Rinse was performed using a buffer which consisted of water, 0.02M Acetate, 0.02M Phosphate, 0.01 M Tris Base, adjusted at pH 5.5 with NaOH 1M
3. AEX chromatography using Eshmuno Q column (from Merck Millipore);
   a) The column was equilibrated using a buffer consisting of water, 0.02M Acetate, 0.02M Phosphate, 0.01 M Tris Base, adjusted at pH 5 with NaOH 1M
   b) The CEX flowthrough was adjusted at pH 5 with acetic acid 1M prior to the load on the column.
   c) A wash buffer was flown through the column, which consisted of water, 0.02M Acetate, 0.02M Phosphate, 0.01 M Tris Base, adjusted at pH 5 with NaOH 1M. The resulting flowthrough (3.b and 3.c) was conserved for analyses;
   d) Regeneration was performed using a buffer which consisted of water, 0.1 M Acetate, 0.1 M Phosphate, 0.05 M Tris Base
   e) Sanitation was performed using NaOH 0.5M
   f) Regeneration was performed using a buffer which consisted of water, 0.02M Acetate, 0.02M Phosphate, 0.01 M Tris Base, 1M NaCl, adjusted at pH 5.5 with NaOH 1M
   g) Rinse was performed using a buffer which consisted of water, 0.02M Acetate, 0.02M Phosphate, 0.01 M Tris Base, adjusted at pH 5 with NaOH 1M

Quality and impurities analysis for the flowthrough of step 3b)c) was performed. The results are presented in table 2.

TABLE 3
A2 purification specifications further
to purification according to method 1

| | Buffer Mix 1 | | | | | |
|---|---|---|---|---|---|---|
| | | | | | | Clipped |
| | Yield | Cumulative | HCP | DNA | HMW | forms |
| Steps | % | yield % | ppm | ppb | % | % |
| Harvest | — | — | 296993 | 1591777 | 1.9 | 9.6 |
| Capture | 98.0 | 95.6 | 100 | 64 | 2.7 | 8.2 |
| CEX | 83.5 | 79.8 | 42 | — | 0.3 | 4.2 |
| AEX | 85.3 | 68.1 | 26 | — | 0.3 | 4.6 |

TABLE 4
A2 purification specifications further to
purification according to method 2

| | Buffer Mix 2 | | | | | |
|---|---|---|---|---|---|---|
| | | | | | | Clipped |
| | Yield | Cumulative | HCP | DNA | HMW | form |
| Steps | % | yield % | ppm | ppb | % | % |
| Harvest | — | — | 217938 | Not yet | N/A | 21.0 |
| Capture | 90.3 | 90.3 | 255 | Not yet | N/A | 19.3 |
| CEX | 87.4 | 78.9 | 150 | — | N/A | 22.3 |
| AEX | 78.4 | 61.9 | 42 | — | 0.3 | 21.0 |

Conclusion: The yield of both purification cascades are comparable (around 65%). Despite the use of different harvest lots, the aggregate percentage and HCP concentration are very low and comparable for both cascades (around 30 ppm of HCP and 0.3% of HMW at the end of the purification). The high value of clipped form in the second example is due to the initial value of the corresponding harvest and a limited impact of the purification on the LMW. To conclude, the different buffer ratio used show great and comparable result in terms of performance and purification.

### REFERENCES

Goyon et al.: Determination of isoelectric points and relative charge variants of 23 therapeutic monoclonal antibodies. Journal of Chromatography B, Volumes 1065-1066, 15 Oct. 2017, Pages 119-128
Baeza-Baeza, J. J., & Garcia-Alvarez-Coque, M. C. (2011). Systematic Approach To Calculate the Concentration of Chemical Species in Multi-Equilibrium Problems”. Journal of Chemical Education, 88(2), 169-173
Carta G. and Jungbauer A. (2010) Protein Chromatography, Wiley-VCH Verlag GMBH.

1-15. (canceled)

16. A method for purifying a protein of interest from a complex solution, said method comprising at least a first liquid chromatography and a second chromatography, wherein for each said liquid chromatography aqueous buffers comprising acetate, phosphate, TRIS base, and optionally sodium hydroxide and/or sodium chloride are used as at least one buffer selected from an equilibration buffer, a loading adjustment buffer, a wash buffer, an elution buffer, a regeneration buffer and a rinse buffer.

17. The method of claim 16, wherein in either the first or the second liquid chromatography, said aqueous buffers are used as at least two buffers selected from an equilibration buffer, a loading adjustment buffer, a wash buffer, an elution buffer, a regeneration buffer and a rinse buffer.

18. The method of claim 16, wherein in the first and in the second liquid chromatography, said aqueous buffers are used as at least two buffers selected from an equilibration buffer, a loading adjustment buffer, a wash buffer, an elution buffer, a regeneration buffer and a rinse buffer.

19. The method of claim 16, wherein in either the first or the second liquid chromatography, said aqueous buffers are used as at least three buffers selected from an equilibration buffer, a loading adjustment buffer, a wash buffer, an elution buffer, a regeneration buffer and a rinse buffer.

20. The method of claim 16, wherein in the first and in the second liquid chromatography, said aqueous buffers are used as at least three buffers selected from an equilibration buffer, a loading adjustment buffer, a wash buffer, an elution buffer, a regeneration buffer and a rinse buffer.

21. The method of claim 16, wherein said aqueous buffers consist of water, acetate, phosphate, TRIS base, and optionally sodium hydroxide and/or sodium chloride.

22. The method of claim 16, wherein said aqueous buffers have the same molar ratio of phosphate over Tris base and the same molar ratio of acetate over phosphate.

23. The method of claim 16, wherein the molar ratio of phosphate over Tris base in said aqueous buffers is between 1.5 and 2.

24. The method of claim 16, wherein the molar ratio of acetate over phosphate in said aqueous buffers is between 1 and 2.5.

25. The method of claim 16, wherein said protein of interest is an antibody, a fragment thereof, or a fusion protein comprising said antibody fragment.

26. The method of claim 16, wherein said protein of interest has an isoelectric point between 6 and 9.5.

27. A method for purifying a protein of interest from a complex solution, wherein said method comprises purifying said complex solution containing said protein of interest through at least a first liquid chromatography and a second chromatography, wherein for each said first and second liquid chromatography, at least one step selected from the group consisting of equilibrating a solid stationary phase, washing a solid stationary phase, eluting the protein of interest, and regenerating a solid stationary phase, is performed using aqueous buffers comprising the compounds acetate, phosphate, TRIS base, and optionally sodium hydroxide and/or sodium chloride.

28. The method of claim 27, wherein said method comprises:
a) purifying said complex solution comprising said protein of interest using at least a first liquid chromatography comprising a first solid stationary phase, thereby obtaining a first protein of interest eluent, wherein at least one step selected from the group consisting of equilibrating said first solid stationary phase, washing said first solid stationary phase, eluting the protein of interest, and regenerating said first solid stationary phase, is performed using aqueous buffers comprising the compounds acetate, phosphate, TRIS base, and optionally sodium hydroxide and/or sodium chloride; and
b) purifying the first protein of interest eluent of step a) using a second liquid chromatography comprising a second solid stationary phase, thereby obtaining a second protein of interest eluent, wherein at least one step selected from the group consisting of equilibrating said second solid stationary phase, washing said second solid stationary phase, eluting the protein of interest, and regenerating said second solid stationary phase, is performed using aqueous buffers comprising acetate, phosphate, TRIS base, and optionally sodium hydroxide and/or sodium chloride.

29. The method of claim 27, wherein in either the first or the second liquid chromatography, at least two steps selected from the group consisting of equilibrating said solid stationary phase, washing said solid stationary phase, eluting the protein of interest, and regenerating said solid stationary phase are performed using aqueous buffers comprising acetate, phosphate, TRIS base, and optionally sodium hydroxide and/or sodium chloride.

30. The method of claim 27, wherein said aqueous buffers consist of water, acetate, phosphate, TRIS base, and optionally sodium hydroxide and/or sodium chloride.

31. The method of claim 27, wherein said aqueous buffers have the same molar ratio of phosphate over Tris base and the same molar ratio of acetate over phosphate.

32. The method of claim 27, wherein the molar ratio of phosphate over Tris base in said aqueous buffers is between 1.5 and 2.

33. The method of claim 27, wherein the molar ratio of acetate over phosphate in said aqueous buffers is between 1 and 2.5.

34. The method of claim 27, wherein said protein of interest is an antibody, a fragment thereof, or a fusion protein comprising said antibody fragment.

35. The method of claim 27, wherein said protein of interest has an isoelectric point between 6 and 9.5.
